(19) United States
(12) Patent Application Publication
(10) Pub. No.: US 2015/0128949 A1
(43) Pub. Date: May 14, 2015

(54) PATIENT INTERFACE DEVICE HAVING HEADGEAR PROVIDING INTEGRATED GAS FLOW AND DELIVERY

(71) Applicant: Gregory John Jablonski, Eindhoven (NL)

(72) Inventor: Gregory John Jablonski, Eindhoven (NL)

(73) Assignee: KONINKLIJKE PHILIPS, EINDHOVEN (NL)

(21) Appl. No.: 13/984,128

(22) PCT Filed: Jan. 31, 2012
(86) PCT No.: PCT/IB12/50448

§ 371 Date: Aug. 7, 2013

Related U.S. Application Data
(60) Provisional application No. 61/441,690, filed on Feb. 11, 2011.

(51) Int. Cl. A61M 16/06; A61M 16/08; A61M 16/00

(52) U.S. Cl. CPC A61M 16/0683; A61M 16/0057; A61M 16/0622; A61M 16/0666; A61M 16/0816; A61M 2209/08
USPC 128/205.25; 128/204.18

(57) ABSTRACT

A patient interface device (8) includes a mask including a interface element (12) structured to engage a portion of the face of a patient and a headgear assembly (14) coupled to the mask. The headgear assembly has a top surface member (28) and a bottom surface member (30), the top surface member being coupled to the bottom surface member to form a fluid delivery housing (20) having an airtight internal chamber (32). The fluid delivery housing is structured to receive a flow of breathing gas from a pressure generating device and is fluidly coupled to the interface element. Also, the headgear assembly has a plurality of spaced support members (34) provided within the internal chamber to define a plurality of flow paths through the internal chamber around the support members, the support members being structured to support the top surface member relative to bottom surface member and prevent the internal chamber from collapsing.

This patent application claims the priority benefit under 35 U.S.C. §119(e) of U.S. Provisional Application No. 61/441,690 filed on Feb. 11, 2011, the contents of which are herein incorporated by reference.

### BACKGROUND OF THE INVENTION

1. Field of the Invention

The present invention relates to respiratory patient interface devices, and, in particular, to a patient interface device having a headgear component that provides an integrated flow path for delivery of breathing gas to the patient.

2. Description of the Related Art

There are numerous situations where it is necessary or desirable to deliver a flow of breathing gas non-invasively to the airway of a patient, i.e., without intubating the patient or surgically inserting a tracheal tube in their esophagus. For example, it is known to ventilate a patient using a technique known as non-invasive ventilation. It is also known to deliver positive airway pressure (PAP) therapy to treat certain medical disorders, the most notable of which is obstructive sleep apnea (OSA). Known PAP therapies include continuous positive airway pressure (CPAP), wherein a constant positive pressure is provided to the airway of the patient in order to splint open the patient's airway, and variable airway pressure, wherein the pressure provided to the airway of the patient is varied with the patient's respiratory cycle. Such therapies are typically provided to the patient at night while the patient is sleeping.

Non-invasive ventilation and pressure support therapies as just described involve the placement of a patient interface device including a mask component having a soft, flexible cushion on the face of a patient. The mask component may be, without limitation, a nasal mask that covers the patient's nose, a nasal cushion having nasal prongs that are received within the patient's nares, a nasal/oral mask that covers the nose and mouth, or a full face mask that covers the patient's face. Such patient interface devices may also employ other patient contacting components, such as forehead supports, cheek pads and chin pads. The patient interface device is connected to a gas delivery tube or conduit and interfaces the ventilator or pressure support device with the airway of the patient, so that a flow of breathing gas can be delivered from the pressure/flow generating device to the airway of the patient. It is known to maintain such devices on the face of a wearer by a headgear having one or more straps adapted to fit over/around the patient's head.

Adherence and compliance to therapy, such as CPAP or other pressure support therapies, is growing to be an industry-wide issue. Factors such as comfort and ease of a patient interface device can greatly affect a patient's adherence and compliance to therapy. Thus, more comfortable, easier to use, and/or simplified designs for patient interface devices are becoming expectations for any product that seeks to compete.

### SUMMARY OF THE INVENTION

In one embodiment, a patient interface device is provided that includes a mask including a cushion structured to engage a portion of the face of a patient and a headgear assembly coupled to the mask. The headgear assembly has a top surface member and a bottom surface member. The top surface member is coupled to the bottom surface member to form a fluid delivery housing having an airtight internal chamber. The fluid delivery housing is structured to receive a flow of breathing gas from a pressure generating device and is fluidly coupled to the cushion to deliver the flow of breathing gas to the cushion. Also, the headgear assembly has a plurality of spaced support members provided within the internal chamber to define a plurality of flow paths through the internal chamber around the support members. The support members are structured to support the top surface member relative to bottom surface member and prevent the internal chamber from collapsing.

In another embodiment, a method a delivering a flow of breathing gas from a pressure generating device to a patient is provided that includes supporting a fluid delivery housing having an airtight internal chamber on the head of a patient. The fluid delivery housing having a top surface member coupled to a bottom surface member and being structured to receive the flow of breathing gas, supporting the top surface member relative to bottom surface member and preventing the internal chamber from collapsing with a plurality of spaced support members provided within the internal chamber. The support members define a plurality of flow paths through the internal chamber around the support members, and communicating the flow of breathing gas along the flow paths and delivering the flow of breathing gas from the internal chamber to an airway of the patient.

These and other objects, features, and characteristics of the present invention, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the invention.

### BRIEF DESCRIPTION OF THE DRAWINGS

FIGS. 1 and 2 are schematic diagrams (side and front views, respectively) of a system adapted to provide a regimen of respiratory therapy to a patient according to one exemplary embodiment of the present invention;

FIG. 3 is a partial exploded isometric view and FIG. 4 is a cross-sectional view taken along lines A-A of FIG. 1 of a fluid delivery housing forming a part of the system of FIGS. 1 and 2 according to one particular, non-limiting embodiment of the present invention; and

FIG. 5 is a partial exploded isometric view of a fluid delivery housing forming a part of the system of FIGS. 1 and 2 according to another particular, non-limiting embodiment of the present invention.

### DETAILED DESCRIPTION OF EXEMPLARY EMBODIMENTS

As used herein, the singular form of “a”, “an”, and “the” include plural references unless the context clearly dictates otherwise. As used herein, the statement that two or more parts or components are “coupled” shall mean that the parts are joined or operate together either directly or indirectly, i.e., through one or more intermediate parts or components, so long as a link occurs. As used herein, “directly coupled” means that two elements are directly in contact with each other. As used herein, “fixedly coupled” or “fixed” means that two components are coupled so as to move as one while maintaining a constant orientation relative to each other.

As used herein, the word “unitary” means a component is created as a single piece or unit. That is, a component that includes pieces that are created separately and then coupled together as a unit is not a “unitary” component or body. As employed herein, the statement that two or more parts or components “engage” one another shall mean that the parts exert a force against one another either directly or through one or more intermediate parts or components. As employed herein, the term “number” shall mean one or an integer greater than one (i.e., a plurality).

Directional phrases used herein, such as, for example and without limitation, top, bottom, left, right, upper, lower, front, back, and derivatives thereof, relate to the orientation of the elements shown in the drawings and are not limiting upon the claims unless expressly recited therein.

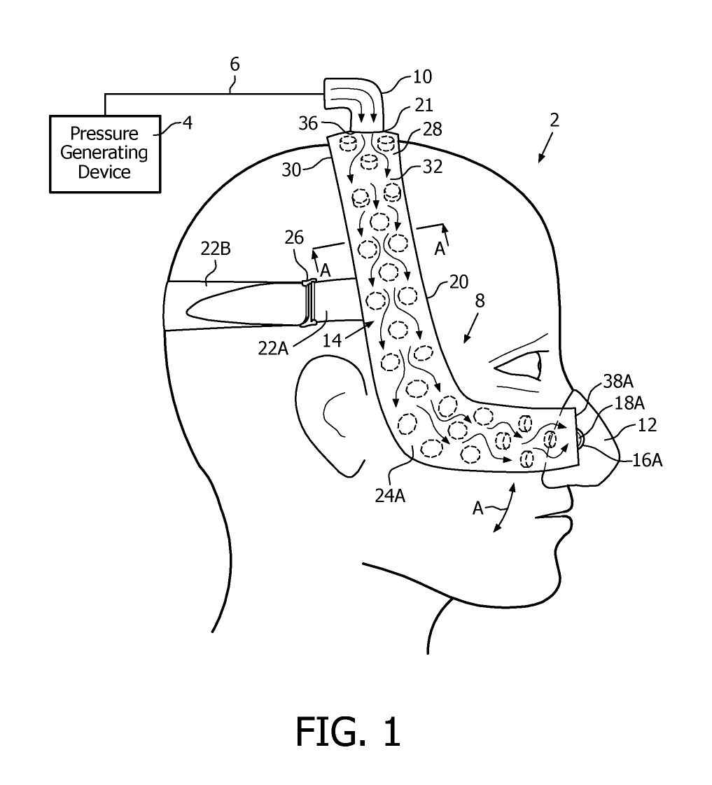

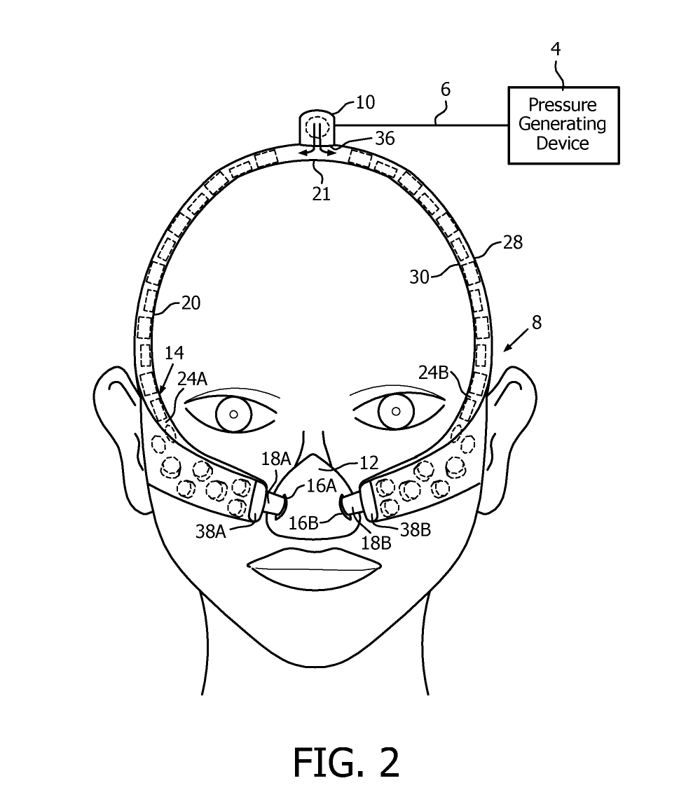

A system 2 adapted to provide a regimen of respiratory therapy to a patient according to one exemplary embodiment is generally shown in FIGS. 1 and 2 (side and front schematic views, respectively). System 2 includes a pressure generating device 4, a patient circuit 6, and a patient interface device 8 having a fluid coupling conduit 10 (e.g. an elbow conduit). Pressure generating device 4 is structured to generate a flow of breathing gas and may include, without limitation, ventilators, constant pressure support devices (such as a continuous positive airway pressure device, or CPAP device), variable pressure devices (e.g., BiPAP®, Bi-Flex®, or C-Flex™ devices manufactured and distributed by Philips Respironics of Murrysville, Pa.), and auto-titration pressure support devices. Patient circuit 6 is structured to communicate the flow of breathing gas from pressure generating device 4 to patient interface device 8, and typically includes a gas delivery conduit or tube coupled to fluid coupling conduit 10.

In the illustrated embodiment, patient interface 8 is a nasal mask type patient interface that covers the nose of a patient. However, other types of patient interface devices, such as, without limitation, a nasal cushion having nasal prongs that are received within the patient's nares, a nasal saddle type cushion structured to be placed against the lower, underside portion of the nose of a patient (wherein the patient's nares are engaged and covered), a nasal/oral mask that covers the nose and mouth, or a full face mask that covers the patient's face, which facilitate the delivery of the flow of breathing gas to, and the removal of a flow of exhalation gas from, the airway of such a patient may be used while remaining within the scope of the present invention.

In the embodiment shown in FIGS. 1 and 2, patient interface 8 includes an interface element 12, which in this exemplary embodiment is a nasal cushion, is fluidly coupled to a headgear assembly 14. Headgear assembly 14, described in greater detail below, is fluidly coupled to fluid coupling conduit 10 and provides a flow path that allows the flow of breathing gas generated by pressure generating device 4 to be communicated to an interior space defined by interface element 12 and then to the airway of a patient. In the illustrated embodiment, the breathing gas is communicated through holes 16A and 16B provided in interface element 12 by way of fluid connectors 18A, 18B. In addition, a frame or faceplate (not shown) may be coupled to interface element 12 to provide support for the interface element. Such a frame or faceplate may be fluidly coupled to interface element 12, and in such an embodiment the holes 16A, 16B may be provided in the frame or faceplate.

Holes 16A and 16B in interface element 12 (or in the frame or faceplate in the alternative embodiment) also allow the flow of exhalation gasses (from the airway of such a patient) to be communicated to an exhaust port (not shown) that may be provided, for example and without limitation, in fluid connectors 18A, 18B or elsewhere on the patient interface device 8. In addition to providing a flow path for breathing gas, headgear assembly 14 is configured to secure patient interface device 8 to the patient's head in a manner wherein interface element 12 is held in place over the nose of the patient so that gasses are permitted to be communicated to and from the airway of the patient through interface element 12

Headgear assembly 14 includes a fluid delivery housing 20, described in greater detail herein, and first and second rear strap members 22A, 22B that extend from fluid delivery housing 20. More specifically, in the illustrated, exemplary embodiment, fluid delivery housing 20 includes a central portion 21 and first and second side portions 24A, 24B extending from central portion 21. As seen in FIGS. 1 and 2, first and second side portions 24A, 24B each generally have an L or dogleg shape. First rear strap member 22A is attached to (e.g., by be sewn thereto) and extends from first side portion 24A, and second rear strap member 22B is attached to (e.g., by be sewn thereto) and extends from second side portion 24B.

As seen in FIGS. 1 and 2, headgear assembly 14 is configured to secure patient interface device 8 to the patient's head when patient interface device 8 is donned by the patient. In particular, when patient interface device 8 is donned by the patient, fluid delivery housing 20 is configured to extend over the top of the patient's head, with the side portions 24A, 24B extending downwardly in front of the patient ears and forwardly over the patient cheeks. Also, second rear strap member 22B is configured to extend around the back the patient's head and couple to first rear strap member 22A.

In the illustrated embodiment, the fit and tension provided by coupled rear strap members 22A, 22B is selectively adjustable by way of a hook and loop fastening system, such as VELCRO®. In particular, the exterior of the end of second rear strap member 22B includes a hook fastener portion, and a corresponding loop fastener portion is provided on the exterior of second rear strap member 22B at a position spaced from the hook fastener portion. In addition, as seen in FIG. 1, first rear strap member 22A includes a looped connecting element 26. Thus, second rear strap member 22B may be threaded through looped connecting element 26 and then bent back on itself in order to adhere the hook fastener portion to the loop fastener portion at a selected location and couple the components together. It will be understood that the illustrated hook and loop fastening arrangement is meant to be exemplary only, and that other selectively adjustable fastening arrangements are also possible within the scope of the present invention.

Fluid delivery housing 20 includes a top surface member 28 and a bottom surface member 30, which are joined to one another along the outer perimeters thereof to form an airtight internal chamber 32. In addition, a plurality of spaced support members 34 are provided within internal chamber 32, with the top surface of each support member 34 being in engagement with the inner side of top surface member 28 and the bottom surface of each support member 34 being in engagement with the inner side of bottom surface member 30. In the exemplary embodiment, each support member 34 is a rigid or semi-rigid structure that provides structural support for fluid delivery housing 20 and prevents internal chamber 32 from collapsing.

In the illustrated embodiment, each support member 34 has a generally cylindrical shape. It will be appreciated, however, that the particular support members 34 shown in FIGS. 1 and 2 are exemplary only and that the support members 34 may have other cross-sectional shapes, such as triangular or rectangular cross-sectional shapes, within the scope of the present invention.

The present invention also contemplates that the pattern and number of support members 34 can be different from that shown in the illustrated embodiments. For example, the support member can be arranged randomly over the area of the top and bottom surface member.

In one particular, non-limiting exemplary embodiment, the width of fluid delivery housing 20 is about 1.250 inches, and each support member is about 0.250 to 0.3125 inches high. In addition, if the support members 35 have a cylindrical shape, they will have a diameter of about 0.375 inches to about 0.500 inches.

Moreover, the present invention further contemplates that the size, shape, and overall geometry of top surface member 28 and bottom surface member 30 can be different from that shown. For example, the present invention contemplates that the top and bottom surface members can be formed in a helmet-like configuration, i.e., covering a relatively large area over the head. Such a configuration is advantageous in that it provides a stable platform for by providing more surface area in contact with the head. Also, is provides more gas flow paths from the point at which the gas is delivered from conduit 6 into the headgear/helmet to the point at which the gas is delivered to nasal interface element 12 or other airway interface element.

Furthermore, an opening 36 is provided in top surface member 28 at central portion 21. Opening 36 is structured to receive fluid coupling conduit 10 such that fluid coupling conduit 10 is fluidly coupled to fluid delivery housing 20, and in particular internal chamber 32. As a result, the flow of breathing gas generated by pressure generating device 4 is able to be delivered to internal chamber 32 of fluid delivery housing 20. In addition, the terminal end 38A, 38B of each of first and second side portions 24A, 24B, respectively, is provided with an opening (not shown) to which a first end of a corresponding fluid connector 18A, 18B may be coupled. As noted elsewhere herein, the other end of each of fluid connector 18A, 18B is coupled to the interior of interface element 12 through openings 16A, 16B. Thus, the flow of breathing gas that is provided to fluid delivery housing 20 by pressure generating device 4 as described above is able to flow through internal chamber 32 along the fluid paths provided around support structures 34 to the interior space defined by interface element 12 and ultimately to the airway of the patient.

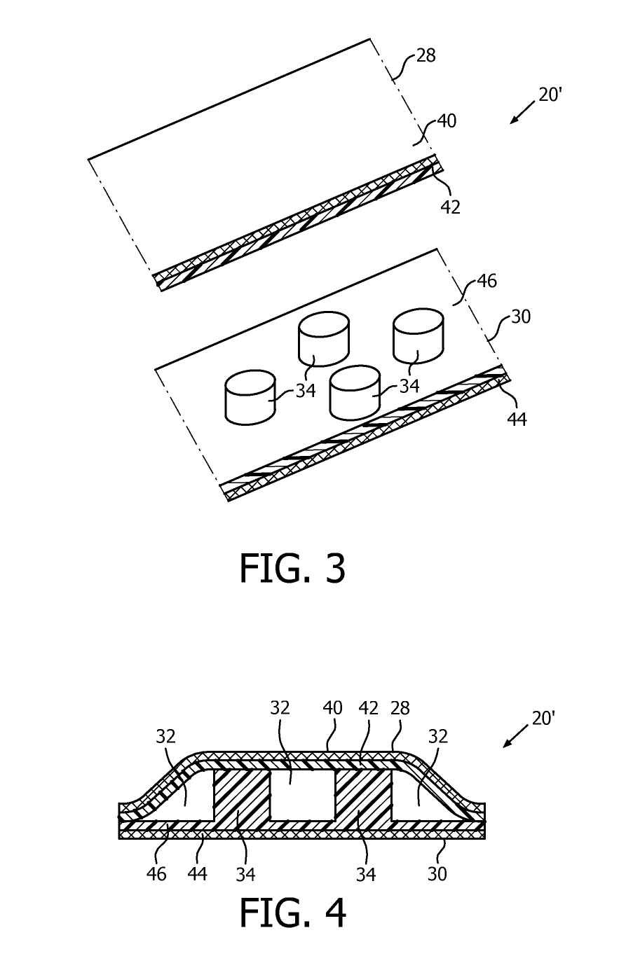

FIG. 3 is a partial exploded isometric view and FIG. 4 is a cross-sectional view taken along lines A-A of FIG. 1 of fluid delivery housing 20 according to one particular exemplary, non-limiting embodiment of the present invention (this particular embodiment is identified by the reference numeral 20′). In the embodiment of FIGS. 3 and 4, top surface member 28 comprises a two layer structure in the form of a urethane backed material. In particular, top surface member 28 includes a fabric layer 40 attached to a urethane layer 42. Similarly, bottom surface member 30 comprises a two layer structure in the form of a urethane backed material and includes a fabric layer 44 attached to a urethane layer 46.

In addition, in this exemplary embodiment, support members 34 are formed as an integral part of urethane layer 46. This may be done in a number of ways, such as, without limitation, a die forming process wherein a urethane material is pulled (e.g., by a vacuum) into a die that forms support members 34, or a molding process wherein a urethane material is shaped in a mold that produces the support members 34. Fluid delivery housing 20′ is then constructed by placing top surface member 28 on bottom surface member 30 and adhering the outer perimeters thereof together, such as by using a die press that melts the urethane material of the two members together or by using a suitable adhesive. Also, during such construction, the top of each support member 34 may be adhered to top surface member 28 by a suitable adhesive or some other suitable process. In an alternative embodiment, each two layer structure is in the form of a silicone backed material. Still other material combinations are possible within the scope of the present invention as long as a suitable seal is formed to produce internal chamber 32.

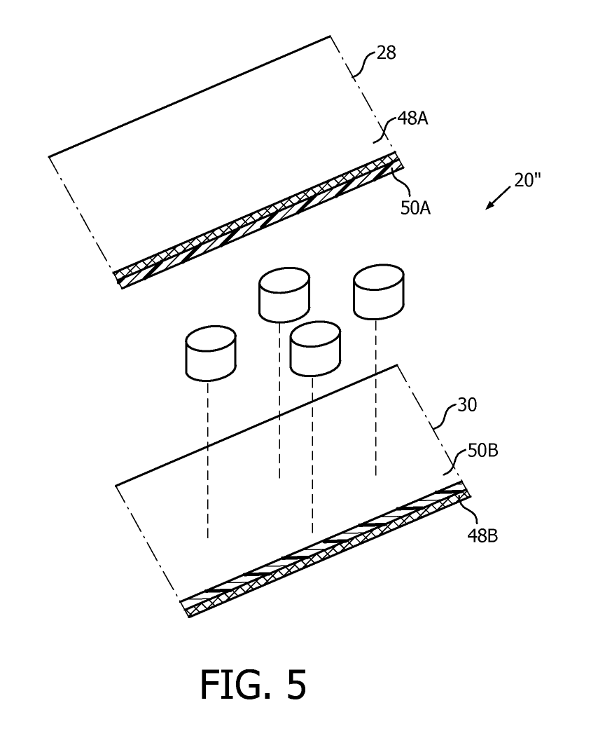

FIG. 5 is a partial exploded isometric view of fluid delivery housing 20 according to another particular exemplary, non-limiting embodiment of the present invention (this particular embodiment is identified by the reference numeral 20″). In the embodiment of FIG. 5, top surface member 28 and bottom surface member 30 are both two layer structures. In the illustrated embodiment, each is a urethane backed material including a fabric layer 48 attached to a urethane layer 50, although other material combinations, such as, without limitation, a silicone backed material, are also possible. In this embodiment, support members 34 are not formed as an integral part of urethane layer 50B, but rather are individual structures that are attached, such as by a suitable adhesive, to urethane layer 50B. The support members 34 in this embodiment are made of a suitable rigid or semi-rigid material, such as, without limitation, foam, urethane or silicone. Fluid delivery housing 20″ is then constructed by placing top surface member 28 on bottom surface member 30 and adhering the outer perimeters thereof together, such as by using a die press that melts the urethane material of the two members together or by using a suitable adhesive. Also, during such construction, the top of each support member 34 may be adhered to top surface member 28 by a suitable adhesive or some other suitable process.

Thus, the designs described herein in the various embodiments provide a comfortable, low profile headgear that also provides for top down delivery of breathing gas to the patient. The comfort and ease of use of such patient interface devices should positively affect a patient's adherence and compliance to therapy. The sidewall of the gas channel are defined by the series of small support structures 34 or “nubs” that allow for a great degree of flexibility for the fluid delivery housing 20 of headgear 14. That is the fluid delivery housing is able to twist and bend, even in a direction generally perpendicular to the longitudinal axis of the gas flow path as indicated by arrow A in FIG. 1, without pinching off the gas flow path. If for example, solid sideways were used, pending in the direction of arrow A would be difficult if not impossible.

In the claims, any reference signs placed between parentheses shall not be construed as limiting the claim. The word “comprising” or “including” does not exclude the presence of elements or steps other than those listed in a claim. In a device claim enumerating several means, several of these means may be embodied by one and the same item of hardware. The word “a” or “an” preceding an element does not exclude the presence of a plurality of such elements. In any device claim enumerating several means, several of these means may be embodied by one and the same item of hardware. The mere fact that certain elements are recited in mutually different dependent claims does not indicate that these elements cannot be used in combination.

Although the invention has been described in detail for the purpose of illustration based on what is currently considered to be the most practical and preferred embodiments, it is to be understood that such detail is solely for that purpose and that the invention is not limited to the disclosed embodiments, but, on the contrary, is intended to cover modifications and equivalent arrangements that are within the spirit and scope of the appended claims. For example, it is to be understood that the present invention contemplates that, to the extent possible, one or more features of any embodiment can be combined with one or more features of any other embodiment.

1. A patient interface device, comprising:
a mask including an interface element structured to engage a portion of a face of a patient; and
a headgear assembly coupled to the mask, comprising:
a top surface member,
a bottom surface member, the top surface member being coupled to the bottom surface member along a perimeter of the top surface member and the bottom surface member to form a fluid delivery housing having an airtight internal chamber, wherein the fluid delivery housing has a central portion and first and second side portions extending from the central portion wherein responsive to the patient interface device being donned by the patient the central portion is configured to rest on a top of the patient's head with the first and second side portions extending downwardly in front of the ears of the patient and forwardly over the cheeks of the patient, wherein the fluid delivery housing is structured to receive a flow of breathing gas from a pressure generating device in the central portion and be fluidly coupled to the interface element to deliver the flow of breathing gas to the interface element, and
a plurality of support members provided within and spaced from one another along an entire length of the fluid delivery housing to define a plurality of flow paths through the internal chamber around all the support members, wherein at all times a top surface of each support member engages an inner side of the top surface member and a bottom surface of each support member engages an inner side of the bottom surface member, and wherein the support members are structured to support the top surface member relative to bottom surface member and prevent the internal chamber from collapsing.

2. The patient interface device according to claim 1, wherein the top surface of each support member is attached to the inner side of the top surface member and the bottom surface of each support member is attached to the inner side of the bottom surface member.

3. The patient interface device according to claim 1, wherein the support members are formed as an integral part of one of the top surface member and the bottom surface member.

4. The patient interface device according to claim 1, wherein the support members are each an individual structure that is attached to at least one of the top surface member and the bottom surface member.

5. The patient interface device according to claim 1, wherein each of the support members has a generally cylindrical shape.

7. The patient interface device according to claim 1, wherein the first and second side portions are each generally L-shaped.

8. The patient interface device according to claim 1, wherein a terminal end of each of the first and second side portions is fluidly coupled to the interface element.

9. The patient interface device according to claim 8, further comprising a first fluid connector coupling the terminal end of the first side portion to the interface element and a second fluid connector coupling the terminal end of the second side portion to the interface element.

10. The patient interface device according to claim 1, wherein the central portion has an opening structured to receive a fluid coupling conduit that is fluidly coupled to the pressure generating device.

11. The patient interface device according to claim 1, wherein the top surface member and the bottom surface member are each multi-layer structures.

12. The patient interface device according to claim 11, wherein the top surface member and the bottom surface member each comprise a first material backed by a fabric material.

13. The patient interface device according to claim 12, wherein the first material is a silicone material or a urethane material.

14. A method a delivering a flow of breathing gas from a pressure generating device to a patient, comprising:
supporting a fluid delivery housing having an airtight internal chamber on a head of a patient, the fluid delivery housing having a central portion and first and second side portions extending from the central portion, wherein the central portion is rests on a top of the patient's head with the first and second side portions extending downwardly in front of the ears of the patient and forwardly over the cheeks of the patient, the fluid delivery housing having a top surface member coupled to a bottom surface member and being structured to receive the flow of breathing gas in the central portion;
supporting the top surface member relative to bottom surface member and preventing the internal chamber from collapsing with a plurality of support members provided within and spaced from one another along an entire length of the fluid delivery housing, the support members defining a plurality of flow paths through the internal chamber around the support members, wherein at all times a top surface of each support member engages an inner side of the top surface member and a bottom surface of each support member engages an inner side of the bottom surface member; and
communicating the flow of breathing gas along the flow paths and delivering the flow of breathing gas from the internal chamber to an airway of the patient.

15. The method according to claim 14, wherein the top surface of each support member is attached to the inner side of the top surface member and the bottom surface of each support member is attached to the inner side of the bottom surface member.
